(19) United States
(12) Patent Application Publication
(10) Pub. No.: US 2021/0236354 A1
(43) Pub. Date: Aug. 5, 2021

(54) INDIVIDUAL FIRST AID KIT WITH INSERT

(71) Applicant: James Cragg, Carson, CA (US)

(72) Inventor: James Cragg, Carson, CA (US)

(21) Appl. No.: 16/778,903

(22) Filed: Jan. 31, 2020

(51) Int. Cl. A61F 17/00; A45C 13/02

(52) U.S. Cl. CPC A61F 17/00; A45C 2011/007; A45C 13/02

(57) ABSTRACT

Disclosed is a first aid kit for wear on a user's personal gear. The first aid kit includes a pouch and an insert placed inside the pouch. The pouch is shaped to prevent catching on objects and includes a set of tabs with a hook and loop fastener, a set of flaps with a hook and loop fastener, and a pair of zippers to the hold the pouch securely closed. A ripcord may be used to release the hook and loop fasteners and open the zippers. A removal strap attached to the insert may be used to withdraw the insert from inside the pouch.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

### 1. Field

The present invention relates to first aid kits, and in particular, first aid kits worn on the person by military personnel and first responders.

### 2. Background

Individual first aid kits are well known in the art. For military and law enforcement personnel, they have been packaged in a variety of formats. First aid kits have been packaged in pouches worn on belts and in dedicated bags. First aid kits have been packed on their own and in conjunction with other equipment. However, in more recent times, as personnel became more acutely aware of the fact that early aid drastically improves the likelihood of survival, there has been a greater emphasis placed on personnel having their own first aid kit packaged in a dedicated standalone or segregated pouch.

Since the preference for standalone first aid pouches became preeminent, first aid pouches have include a design tradeoff between access and security. That is, some designs included great security in that the contents of the pouch were unlikely to be accidently removed. Other designs sacrificed some security for access and the ability to bring the contents of the first aid kit rapidly to bear in an emergent situation. Primarily, concern focused on occasions when personnel were either entering or exiting a vehicle. Because the first aid kids were generally positioned near a the small of a user's back, the movements executed when mounting or dismounting a vehicle could cause the first aid kit to become entangled with various components in and around a vehicle's seat. The entanglement could cause the pouch to open and the contents to spill or be removed. Designs which were secure against this risk were also more difficult to open when the contents were needed, and designs with greater access accepted some risk that the entanglement could cause spills or removals in exchange for better access when needed. However, if the contents of the first aid kit were lost without the knowledge of the user, and later needed, the result could be catastrophic. The required tradeoffs made designs on both ends of the security and access spectrums less than ideal.

For the foregoing reasons, there is a need for a first aid kit which includes security against accidental removal of the contents, but allows a user immediate access when required.

### BRIEF SUMMARY

Disclosed herein is a first aid kit for wear on the person. The first kid may include a pouch. The pouch may include a rear panel having a rear panel top edge, a rear panel bottom edge, a rear panel first vertical edge, a rear panel second vertical edge, a rear panel exterior surface and a rear panel interior surface. The pouch may further include a first opposing side wall. The first opposing side wall may have a first straight edge and a first curved edge, the first straight edge may be connected to the rear panel top edge. The pouch may further include a second opposing side wall. The second opposing side wall may have a second straight edge and a second curved edge. The second straight edge may be connected to the rear panel bottom edge. The pouch may further include a front panel. The front panel may include a front panel top edge, a first front panel vertical edge, a front panel bottom edge, a second front panel vertical edge, a front panel exterior surface, and a front panel interior surface. The front panel interior surface may include a first portion of a first hook and loop fastener. The front panel top edge may be connected, at least in part, to the first curved edge. The first front panel vertical edge may be connected to the rear panel first vertical edge, and the front panel bottom edge may be connected, at least in part, to the second curved edge. The pouch may further include a first zipper. The first zipper may include a first tape portion and a second tape portion. The first tape portion may be connected to the front panel top edge, and the second tape portion may be connected to the first curved edge. The pouch may further include a second zipper. The second zipper may include a first tape strip and a second tape strip. The first tape strip may be connected to the front panel bottom edge, and the second tape strip may be connected to the second curved edge. An opening may be defined by the front panel second vertical edge, the rear panel second vertical edge, the first zipper, and the second zipper. The pouch may further include an interior flap connected to the rear panel interior surface. The interior flap may extend exterior of the opening. The interior flap may include a second portion of the first hook and loop fastener. The second portion may be removably attached to the first portion of the first hook and loop fastener. The pouch may further include an interior end tab. The interior tab may be located, at least in part, between the interior flap and the rear panel. The interior tab may extend exterior of the opening. The interior end tab may include a first portion of a second hook and loop fastener. The pouch may further include an exterior end tab. The exterior end tab may be attached to the front panel along a single edge of the exterior end tab. The exterior end tab may include a second portion of the second hook and loop fastener. The second portion may be removably attached to the first portion of the second hook and loop fastener. The pouch further including a ripcord. The ripcord may be attached to the front panel between the exterior end tab and the second vertical edge. At least a portion of the ripcord may be removably placed between the first and second portions of the second hook and loop fastener. The first aid kid may also include an insert. The insert may be removably placed in the interior of the pouch. The insert may include a rigid plate. The insert may also include a removal strap. The removal strap may have an attached end, which may be attached to the rigid plate. The removal strap may also have a free end, which may include a buckle. When a user pulls the ripcord, the pulling motion may separate the first and second portions of the second hook and loop fastener, the first and second portions of the first hook and loop fastener, and may open the first zipper and the second zipper, providing access to the insert. Access to the insert may allow the user to grasp the removal strap and may allow the user to withdraw the insert through the opening.

Further disclosed is a method for manufacturing a first aid kit. The method may include forming a pouch. The pouch may include a rear panel, and a front panel. The front panel may include a first portion of a first hook and loop fastener. The pouch may further include an interior flap attached to the rear panel. The interior flap may include a second portion of the first hook and loop fastener. An exterior end tab may be attached to the front panel. The exterior end tab may include a first portion of a second hook and loop fastener. The pouch may further include an interior end tab. The interior end tab may include a second portion of the second hook and loop fastener. The pouch may further include a first zipper which may be attached to a first edge of the front panel, and a second zipper which may be attached to a second edge of the front panel. The front panel, the rear panel, the first zipper, and the second zipper may define an opening in the pouch. The method may further include inserting an insert in the opening. The insert may include a removal strap. The method may further include closing the first zipper and the second zipper. The method may further include attaching the first portion of the first hook and loop fastener to the second portion of the first hook and loop fastener. The method may further include attaching the first portion of the second hook and loop fastener to the second portion of the second hook and loop fastener.

Further disclosed is a first aid kit for rapid access. The first aid kit may include a pouch. The pouch may include a rear panel, a first opposing side, and a second opposing side. Each of the opposing sides may include a first edge and a second edge. Each of the first and second opposing sides may be attached to the rear panel along the first edge. A front panel may include a first portion of a first hook and loop fastener. The front panel may be attached, at least in part, to each of the first and second opposing sides along their respective second edges. The rear panel, the first and second opposing sides, and the front panel may form an interior. The pouch may further include an interior flap attached to the rear panel. The interior flap may include a second portion of the first hook and loop fastener. The second portion of the first hook and loop fastener may be removably attached to the first portion of the first hook and loop fastener. The pouch may further include an interior end tab. The interior end tab may be attached to the rear panel and the interior flap. The interior end tab may include a first portion of a second hook and loop fastener. The pouch may further include an exterior end tab. The exterior end tab may be attached along a single edge of the exterior end tab to the front panel. The exterior end tab may include a second portion of the second hook and loop fastener. The second portion of the second hook and loop fastener may be removably attached to the first portion of the second hook and loop fastener. The pouch may further include a first zipper. The first zipper may include a first tape portion and a second tape portion. The first tape portion may be connected to the front panel, and the second tape portion may be connected along the second edge of the first opposing side. The pouch may further include a second zipper. The second zipper may include a first tape strip and a second tape strip. The first tape strip may be connected to the front panel, and the second tape strip may be connected along the second edge of the second opposing side. The first aid kit may further include an insert. The insert may be removably placed in the interior. The insert may include a removal strap, which may be used for withdrawing the insert from the interior.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows a perspective view of the first aid kit with the rip cord draped over a portion of the second hook and loop fastener;

FIG. 2 shows a perspective view of the open second hook and loop fastener;

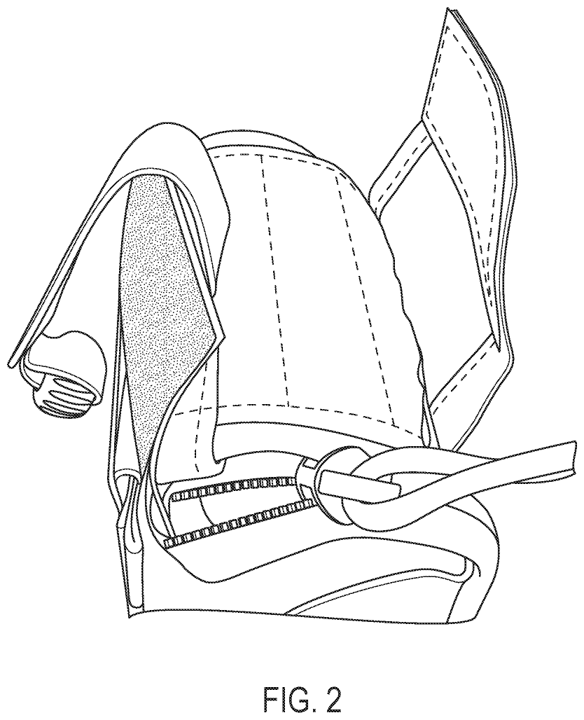

FIG. 3 shows a perspective view of the first aid kit with both the first and second hook and loop fasteners opened by the rip cord;

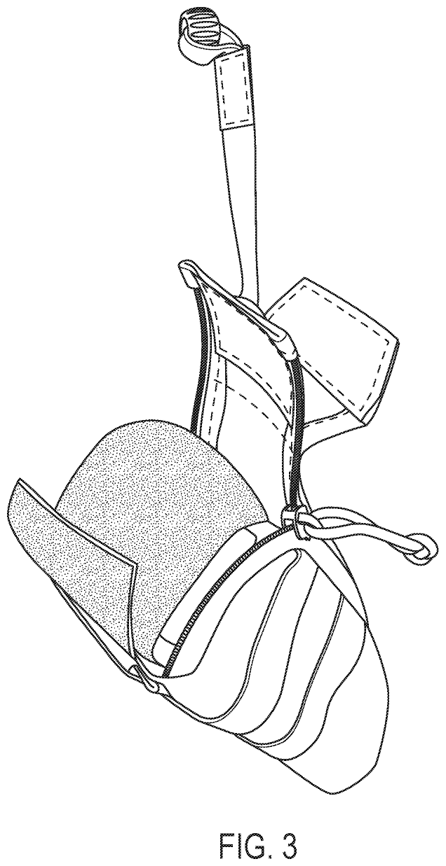

FIG. 4 shows a perspective view of the front panel and interior flap rotated away to reveal the insert with a removal strap extended;

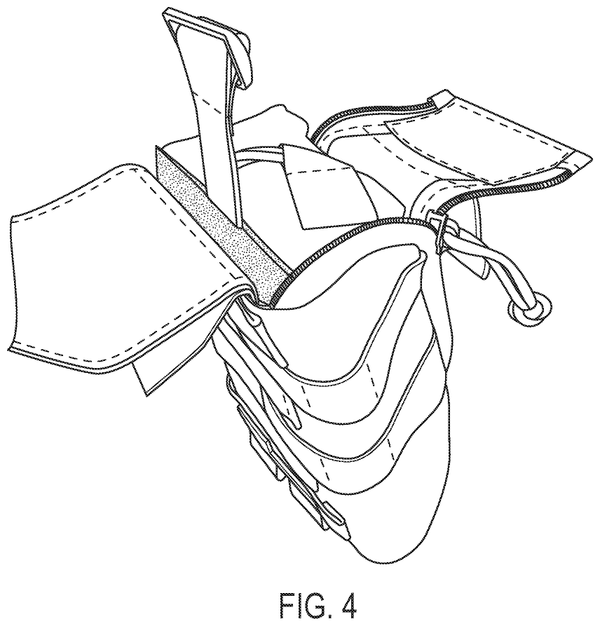

FIG. 5 shows a perspective view of the first aid kit with the insert partially removed; and

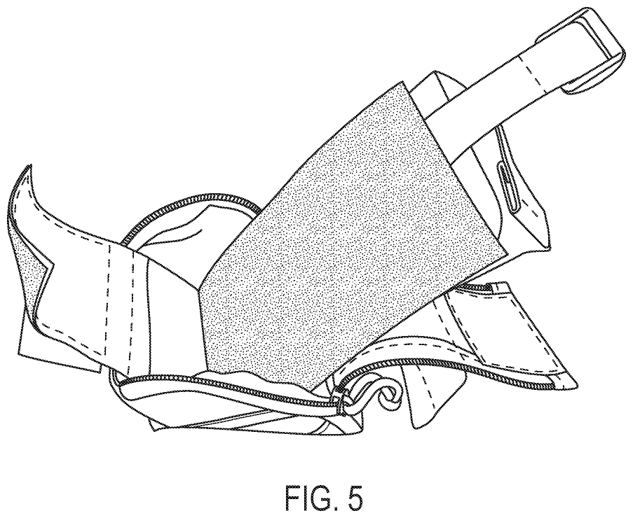

FIG. 6 shows a perspective view 180 degrees of that of FIG. 5 of the first aid kit with the insert partially removed.

### DETAILED DESCRIPTION

The detailed description set forth below in connection with the appended drawings is intended as a description of the presently preferred embodiment of a first aid kit, and is not intended to represent the only form in which it can be developed or utilized. The description sets forth the functions for developing and operating the system in connection with the illustrated embodiments. It is to be understood, however, that the same or equivalent functions may be accomplished by different embodiments that are also intended to be encompassed within the scope of the present disclosure. It is further understood that the use of relational terms such as first, second, distal, proximal, and the like are used solely to distinguish one from another entity without necessarily requiring or implying any actual such relationship or order between such entities.

Disclosed is a first aid kit. The first aid kit is particularly adapted to be carried on standardized military and first responder gear. The first aid kit includes a pouch. The pouch may include a web of attachment straps on a rear panel of the pouch. The web of attachment straps may be adapted to accommodate a plurality of attachment systems. Further, the web of attachment straps may be adapted to prevent slipping on some carriers, for example, belts.

On an opposite side, the pouch may further include a system of tabs and flaps. A set of tabs provide security for the closure of the pouch. The set of tabs may include an exterior end tab and an interior end tab. The set of tabs may be held secure by hook and loop fastener. The hook and loop fastener may be sized to cover a ripcord which is secured in between the hook portion and the loop portion of the fastener. When a user pulls the ripcord, the motion of the pulling of the ripcord separates, at least in part, the hook and loop fastener, thereby separating, at least in part, the exterior end tab from the interior end tab.

As the user continues to pull the ripcord, the motion completes the separation of the set of tabs and affects a set of flaps. The set of flaps includes a front panel and an interior flap. The front panel is secured by zippers on opposing edges, and to an interior flap by a first portion of a hook and loop fastener. The first portion is located near an end portion of the front panel. The second portion of the hook and loop fastener is located on an outer surface of the interior flap. The continued motion of the ripcord separates the hook and loop fastener portion on the front panel from that on the interior flap, and opens the zippers along the opposing edges of the front panel.

Once the zippers are opened by the user, the user may access an interior of the pouch. The user may grasp a removal strap which has a buckle attached to a free end, and pull through the opening created by the disengagement of the set of tabs and the set of flaps. Through the pulling motion, the user may withdraw an insert from the interior of the pouch. The insert may carry a predetermined load out of first aid supplies.

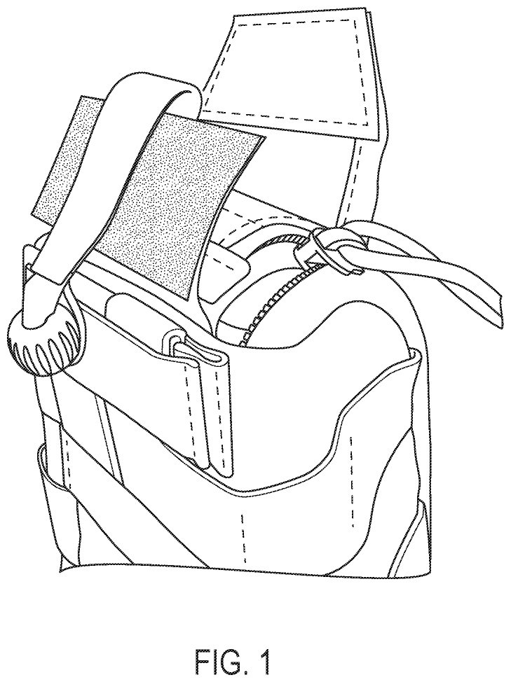

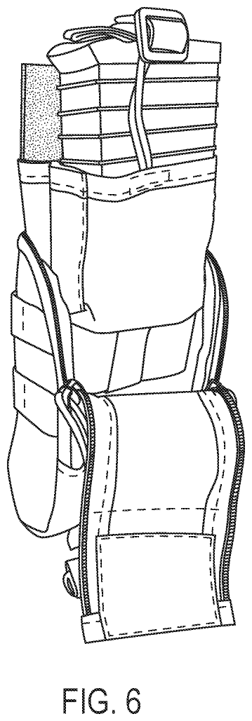

More specifically describing the structure of the first aid kit 100, as shown in FIGS. 1 and 6, the first aid kit 100 may include the pouch 102. The pouch 102 may include an outer shell 104. The outer shell 104 may include a rear panel 106, a front panel 108, and two opposing side panels 110a, 110b which mirror one another. The side panels 110a, 110b may include a first edge 112a, 112b with rounded portions 114a, 114b adjacent to both of a first end 116a, 116b and a second end 118a, 118b of the pouch 102. The side panels 110a, 110b may further include a second edge 120a, 120b which is substantially straight for the length of the second edge 120a, 120b. The first edge 112a, 112b may connect to the second edge 120a, 120b at the first end 116a, 116b, and the first edge 112a, 112b may connect with the second edge 120a, 120b at the second end 118a, 118b.

A top edge 124 of the front panel 108 may be connected to the first edge 114a of a first side panel 110a of the two opposing side panels 110a, 110b, and a bottom edge 126 of the front panel 108 may be connected to the first edge 114b of the second of two opposing side panels 110b. The connection may be made by stitching the edge 124, 126 of the front panel 108 and the edge 114a, 114b of the corresponding side panel 110a, 110b together. Alternatively, the connection may be made by adhesives, or mechanical fasteners or by any method known in the art which will connect the front panel 108 and opposing side panels 110a, 110b along the length to be connected. Because of the connection to both of the two opposing side panels 110a, 110b along each of the first edges 114a, 114b, the front panel 108 includes curved or rounded ends. This configuration provides the advantage of making it less likely that the first aid kit 100 will get snagged on external equipment. Users are likely to wear the first aid kit 100 to the rear of their person, at or near the beltline. For example, a user may wear the first aid kit 100 in the small of the user's back. As a user slides in or out of a vehicle, the rear beltline portion of their body makes contact with one or more portions of the vehicle. Where the first aid kit 100 to have additional panels connecting and located between the rear panel 106 and the front panel 108, such additional panels would present more surface area while the user moved laterally in and out of a vehicle. This increase in surface area makes it more likely that such a first aid kit would become entangled with, or blocked by, portions of the vehicle interior. Because of the curved or rounded ends of the opposing side panels 110a, 110b and connected front panel 108, the first aid kit 100 is more likely to slide off of contact with portions of the vehicle interior, preventing entanglement or blockage. This aids in smooth mounting and dismounting from the vehicle, which may be critical in emergent conditions.

A top edge zipper 128 may be interposed between the connection of top edge 124 of the front panel 108 and the first edge 114a of the first of the opposing side panels 110a for at least a portion of the connection's length. Thus, the top edge 124 of the front panel 108 and the first edge 112a of the first of the opposing side panels 110a may each connect to opposing sides 130a, 130b of the tape 132 of the top edge zipper 128. The top edge zipper 128 may be substantially half the length of the connection of the opposing side panel 110a and the front panel 108. Alternatively, the top edge zipper 128 may be less than half the length of the connection or more than half the length of the connection.

A bottom edge zipper 134 may be interposed between the connection of bottom edge 126 of the front panel 108 and the first edge 112b of the second of the opposing side panels 110b for at least a portion of the connection's length. Thus, the bottom edge 126 of the front panel 108 and the first edge 114b of the second of the opposing side panels 110b may each connect to opposing sides 136a, 136b of the tape of the bottom edge zipper 134. The bottom edge zipper 134 may be substantially half the length of the connection. Alternatively, the bottom edge zipper 134 may be less than half the length of the connection or more than half the length of the connection.

Each of the top edge zipper 128 and the bottom edge zipper 134 may have a looped cord 138a, 138b placed through the slider 140a, 140b of the zipper 128, 134. This makes the slider 140a, 140b easier for a user to grasp and operate should it become necessary to do so. Alternatively, the traditional metal tab may be used on the slider 140a, 140b. However, grasping the slider 140a, 140b directly is not the way the zippers are normally operated with the first aid kit 100 as will be described in greater detail below.

A top edge 142 of the rear panel 106 may connect to the second edge 120a of one of the two opposing sides 110a, and a bottom edge 144 of the rear panel 106 may connect to the second edge 120b of the second of the two opposing sides 110b. Additionally, a first vertical edge 146 of the rear panel 106 may be connected to a first vertical edge 148 of the front panel 108. However, the second vertical edge 150 of the rear panel 106 does not similarly connect to the second vertical edge 152 of the front panel 108. Rather, these edges 150, 152 are placed in a different configuration in order to provide a user access in to the interior 154 of the pouch 102, as described below.

An insert 156 may be placed in the interior 154 of the pouch 102. The insert 156 may be of any dimensions which will allow the insert 156 to fit within the pouch 102 and to be withdrawn therefrom by a user. The insert 156 may include various compartments and straps to ensure that all the medical supplies carried by the insert 156 stay contained in, or held in place on, the insert 156 as the insert is withdrawn from the pouch 102. For example, the insert 156 may have a rigid plate 158 onto which various components are attached. The rigid plate 158 may have a shape generally corresponding to that of the rear panel 106, but, as noted above, with slightly smaller dimensions. The rigid plate 158 may have a first surface 160 which faces the rear panel 106 when the insert 156 is inserted in to the pouch 102, and a second surface 162 which faces the front panel 108 when the insert 156 is inserted in the pouch 102. The rigid plate 158 may be of a plastic, carbon fiber, aluminum, or any material which is rigid enough to hold the various components attached to it in fixed special relationships, and to hold its shape when inserted and removed from the pouch 102.

The first surface 160 of the rigid plate 158 may include a first piece 164 of a hook and loop fastener positioned substantially covering the first surface 160 of the rigid plate 158. The first piece 164 of hook and loop fastener may be attached using sewing or adhesives or any other method known in the art to permanently attach the first piece 164 to the rigid plate 158. The first piece 164 may connect to a second piece 166 of hook and loop fastener placed on the interior surface of the rear panel 106. The connection of the first piece 164 and the second piece 166 hold the insert 156 in place, even if the pouch 102 is opened. The connection of the first piece 164 and the second piece 166 therefore provides an additional security for the insert 156, and helps prevent inadvertent removal of the insert 156.

The insert 156 may further include a central compartment 168, which is substantially cube shaped, with a single side missing. The missing side defines an opening 170 to an interior of the central compartment 168. The central compartment 168 may be made of a nylon or other material, and the panels or sides of the central compartment 168 may be sewn together or may be created by folding a single piece of material to create two or more of the sides or panels. The opening 170 may be oriented on the rigid plate 158 so that the opening 170 is facing the opening in the pouch 102. The central compartment 168 may include a corded strap 172 which is attached on both ends to the interior of the central compartment 168. The ends may be attached to the interior of the central compartment 168 using hook and loop fastener so that the ends of the corded strap 172 may be detached and the corded strap 172 pulled entirely free of the central compartment 168. Alternatively, the corded strap 172 may be slipped on and off medical supplies which extend outward from the central compartment 168, in order to hold the medical supplies in place.

On an opposite side of the central compartment 168, the insert 156 may include a hold down strap 174. The hold down strap may have an attached end 176 and a free end 178. The attached end 176 of the hold down strap 174 may attach to a side or panel of the central compartment 168 opposite the opening 170 of the central compartment 168. Alternatively, the attached end 176 of the hold down strap 174 may be attached to the rigid panel 158. The attached end 176 of the hold down strap 174 may be attached using sewing or adhesives or any other method known in the art to create a permanent attachment. The free end of the hold down strap may have a first piece of a hook and loop fastener attached. The rigid plate 158 may have a second piece of the hook and loop fastener attached. The second piece may be attached to the first surface or the second surface of the rigid plate 158, with the first surface being preferred. The first and second piece of hook and loop fastener are positioned, respectively, on the free end portion and the rigid plate 158 to best hold medical supplies between the hold down strap 174 and the rigid plate 158 by compressing the supplies between the hold down strap 174 and the rigid plate 158.

The insert 156 may further include a removal strap 180. The removal strap 180 may be attached to an end portion 182 of the rigid plate 156 which is closest to the opening of the pouch 102. The removal strap 180 may extend from the rigid plate 156 beyond the opening of the pouch 102 when the pouch 102 is opened. The removal strap 180 may have a buckle 184 attached to a free end portion 186 of the removal strap 180. The buckle 184 may be attached to the free end portion 186 by folding the removal strap 180 back on to itself and sewing the removal strap to itself so as to form a loop containing a portion of the buckle 184. The buckle 184 will allow a user to more easily grasp the removal strap 180 when withdrawing the insert 156 from the interior of the pouch 102. The removal strap 180 may be made of a more rigid material than the other straps on the pouch 102, and may be doubled or tripled by sewing layers of the removal strap 180 on to each other. This may give the removal strap 180 additional rigidity so that it extends away from the insert 156, making it easier for a user to find and grasp.

Once the insert 156 is placed inside the pouch 102, the hook and loop fastener on the first surface 160 of the rigid plate 158 and the interior surface of the rear panel 106 engage, holding the insert 156 in a substantially fixed position in the interior 154 of the pouch 102. The removal strap 180 may then be folded over, so that the removal strap 180 is to the interior 154 of the pouch 102, and the opening of the pouch 102 may be closed and secured.

Near the second vertical edge 150 of the rear panel 106, an interior flap 188 may be attached to the interior surface of the rear panel 106. The interior flap 188 may be attached so that a portion of the interior flap 188 extends beyond the second vertical edge 150 of the rear panel 106. The portion 190 of the interior flap 188 that extends past the second vertical edge 150 has two surfaces, a rear-facing surface 192 and a front facing surface 194. A first portion 196 of a hook and loop fastener is attached to the rear-facing surface 192. A second portion 198 of a hook and loop fastener is attached to the second vertical edge 152 portion of the front panel 108.

A user may pull a rip cord 200, which is attached to the end portion near the second vertical edge 152 of the front panel 108. The ripcord 200 may include a cord portion 202 and an end portion 204. The end portion 204 may be enlarged so that when a user grasps the ripcord 200, the user's hand does not inadvertently slide off an end of the ripcord 200.

Pulling on the ripcord 200 in either a direction toward the opening of the pouch 102 or away from the opening of the pouch 102 will drag the zippers 128, 134 in that direction, either opening or closing both zippers 128, 134. The interior flap 188 may be folded back so the interior flap 188 is in an interior of the pouch 102 with the hook and loop fastener facing the exterior. A user may pull the looped cords 138a, 138b to close the zippers 128, 134. As the zippers 128, 134 near being closed, the hook and loop second portion 198 on the second vertical edge 152 portion of the front panel 108 may be attached to the first portion 196 on the interior flap 188, holding the front panel 108 closed. The zippers 128, 134 may then be zipped completely to the end of their travel to further hold the front panel 108 closed.

An interior end tab 206 may be attached to the rear panel 106 so a portion of the interior end tab 206 is between the rear panel 106 and the portion of the interior flap 188 also attached to the rear panel 106. The interior end tab 206 may have an exterior surface 208 and an interior surface 210. The interior end tab 206 also extends from the opening of the pouch 102. The interior end tab 206 is similar in configuration to the interior flap 188, including the attachment of a first portion 212 of a hook and loop fastener, but with the exception that the interior end tab 206 is shorter, and therefore does not extend as far from the pouch 102.

An exterior end tab 214 may be attached along a single edge 216 near an end portion of the front panel 108. The edge 216 along which the exterior end tab 214 is preferably attached is the vertical edge furthest from the second vertical edge 152 of the front panel 108. This attachment configuration allows the exterior end tab 214 to rotate around the attachment edge 216, similar to the way a door swings on its hinges. The exterior end tab 214 has an exterior surface 218 and an interior surface 220. A second portion 222 of the hook and loop fastener may be attached to the interior surface 220.

The interior end tab 206 is folded back so the interior surface 210 abuts the front panel 108 and the first portion 212 of the hook and loop fastener facing outward. The ripcord 200 may be partially coiled and placed on top of the first portion 212 of the hook and loop fastener, with a free end 224 of the ripcord 200 extending from an edge of the first portion 212 of the hook and loop fastener. The free end 224 of the ripcord 200 may include a ball structure, or substantially similar structure, which allows for easier grasping of the ripcord 200 and prevents a user's hand from slipping off the end of the ripcord 200. Then, the exterior end tab 214 is rotated down on to the interior end tab 206, sandwiching the ripcord 200 between the first portion 212 and the second portion 222 of the hook and loop fasteners. This secures the first aid kit 100, and the first aid kit 100 is ready to be placed in to operation.

In operation, the first aid kit 100 may be placed on equipment worn by a user. The first aid kit 100 may be attached to a belt, to a vest, to another bag or pouch, or anywhere a user finds acceptably accessible. The pouch 102 may include a web 226 of attachment straps on a rear panel 106 of the pouch 102. The web 226 of attachment straps may be adapted to accommodate a plurality of attachment systems. Further, the web 226 of attachment straps may be adapted to prevent slipping by the addition of cross straps or by weaving the attachment straps to increase friction between the attachment straps and the elements to which the first aid kit 100 is being attached.

A user may reach for the first aid kit 100. Either by sight or by feel, a user may find the ripcord 200. Grasping the ripcord 200, a user may pull the ripcord 200 in a substantially curving pattern. The curving pattern first causes the release of the set of tabs 206, 214 and the set of flaps 108, 188, and then opens the zippers 128, 134 as will be described in further detail below. When a user pulls the ripcord 200, the motion of the pulling of the ripcord 200 separates, at least in part, the hook and loop fasteners 212,222 of the set of tabs 206, 214. Thus, this initial motion unfastens, at least in part, the exterior end tab 214 from the interior end tab 206. Specifically, the motion of the ripcord 200 is away from the front panel 108, and causes the exterior end tab 214 to separate and rotate away from the interior end tab 206. The exterior end tab 214 rotates around the attachment edge 216 as is described in detail above.

As the user continues to pull the ripcord 200, the motion completes the separation of the set of tabs 206, 214 and affects a set of flaps 108, 188. The set of flaps 108, 188 includes the front panel 108 and the interior flap 188. As the user continues to pull on the ripcord 200, the ripcord 200 goes taut and is placed under tension. The ripcord 200 peels back the front panel 108 as the ripcord 200 is pulled. Simultaneously, the hook and loop fasteners 196, 198 between the front flap 108 and the interior flap 188 separates, and the top edge zipper 128, and the bottom edge zipper 134, collectively the zippers 128, 134, begin to open. The continued motion of the ripcord 200 separates the hook and loop fastener 198 portion on the front panel 108 from the hook and loop portion 196 on the interior flap 188 because the pulling of the ripcord 200 causes the front panel 108 to peel away from the interior flap 188, with the front panel 108 both rotating and translating away. The zippers 128, 134 begin to open because the two sets of teeth elements are pulled toward the two sides of the sliders 140a, 140b. This separation causes the sliders 140a, 140b to move away from the separating teeth elements and on to the still closed elements. These next elements are then separated, and the process continues.

Once the zippers 128, 134 are completely opened by the user, the user may access an interior 154 of the pouch 102. The user may grasp the removal strap 180 at any point, including the buckle 184 attached to the free end 186, and pull the insert 156 through the opening created by the disengagement of the set of tabs 206, 212 and the set of flaps 108, 188. As the user pulls on the insert 156, the hook and loop fastener 164, 166 holding the rigid plate 158 to the rear panel 106 is separated, and the insert 156 moves freely. Through the pulling motion, the user may withdraw the insert 156 from the interior 154 of the pouch 102. Once the insert 156 is removed, the user may access the medical supplies in and on the insert 156. The user may detach or slide off the corded strap 172, or may detach the hold down strap 174 to gain access to the medical supplies. The rigid plate 158 may be placed on a surface so that it is between the medical supplies and the surface to prevent contamination of the medical supplies from bacteria on the surface. The rigid plate 158 further helps to stabilize the insert 156 by preventing it from rolling over.

The above description is given by way of example, and not limitation. Given the above disclosure, one skilled in the art could devise variations that are within the scope and spirit of the invention disclosed herein, including various ways of defining the commands in the protocol. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A first aid kit for wear on the person, comprising:
a pouch including:
a rear panel having a rear panel top edge, a rear panel bottom edge, a rear panel first vertical edge, a rear panel second vertical edge, a rear panel exterior surface and a rear panel interior surface
a first opposing side wall having a first straight edge and a first curved edge, the first straight edge being connected to the rear panel top edge;
a second opposing side wall having a second straight edge and a second curved edge, the second straight edge being connected to the rear panel bottom edge;
a front panel, the front panel including a front panel top edge, a first front panel vertical edge, a front panel bottom edge, a second front panel vertical edge, a front panel exterior surface, and a front panel interior surface including a first portion of a first hook and loop fastener, the front panel top edge connected, at least in part, to the first curved edge, the first front panel vertical edge connected to the rear panel first vertical edge, and the front panel bottom edge connected, at least in part, to the second curved edge;
a first zipper including a first tape portion and a second tape portion, the first tape portion connected to the front panel top edge, and the second tape portion connected to the first curved edge;
a second zipper including a first tape strip and a second tape strip, the first tape strip connected to the front panel bottom edge, and the second tape strip connected to the second curved edge;
an opening defined by the front panel second vertical edge, the rear panel second vertical edge, the first zipper, and the second zipper;
an interior flap connected to the rear panel interior surface and extending exterior of the opening, the interior flap including a second portion of the first hook and loop fastener removably attached to the first portion of the first hook and loop fastener;
an interior end tab located, at least in part, between the interior flap and the rear panel and extending exterior of the opening, the interior end tab including a first portion of a second hook and loop fastener;
an exterior end tab attached to the front panel along a single edge of the exterior end tab, the exterior end tab including a second portion of the second hook and loop fastener removably attached to the first portion of the second hook and loop fastener; and
a ripcord attached to the front panel between the exterior end tab and the second vertical edge, and at least a portion of the ripcord removably placed between the first and second portions of the second hook and loop fastener;
an insert removably placed inside the pouch including:
a rigid plate; and
a removal strap having an attached end, which is attached to the rigid plate, and a free end, the free end including a buckle;
wherein, when a user pulls the ripcord, the pulling motion separates the first and second portions of the second hook and loop fastener, the first and second portions of the first hook and loop fastener, and opens the first zipper and the second zipper, providing access to the insert; and
allowing the user to grasp the removal strap and to withdraw the insert through the opening.

2. The method of claim 1, wherein the rigid place includes a first portion of a third hook and loop fastener.

3. The method of claim 2, wherein the rear panel includes a second portion of a third hook and loop fastener.

4. The method of claim 3, wherein when a user pulls the removal strap, the insert translates through the opening.

5. The method of claim 4, wherein when a user pulls the removal strap, the first portion and the second portion of the third hook and loop fastener separate.

6. The method of claim 1, wherein the insert further includes a central compartment.

7. The method of claim 1, wherein the rear panel exterior surface includes a plurality of attachment straps.

8. A method for manufacturing a first aid kit, comprising:
forming a pouch, the pouch including a rear panel, a front panel including a first portion of a first hook and loop fastener, an interior flap attached to the rear panel, the interior flap including a second portion of the first hook and loop fastener, an exterior end tab attached to the front panel and including a first portion of a second hook and loop fastener, an interior end tab including a second portion of the second hook and loop fastener, a first zipper attached to a first edge of the front panel, and a second zipper attached to a second edge of the front panel, the front panel, the rear panel, the first zipper, and the second zipper defining an opening in the pouch;
inserting an insert in the opening, the insert including a removal strap;
closing the first zipper and the second zipper;
attaching the first portion of the first hook and loop fastener to the second portion of the first hook and loop fastener; and
attaching the first portion of the second hook and loop fastener to the second portion of the second hook and loop fastener.

9. The system of claim 8, wherein the rear panel further includes a first portion of a third hook and loop fastener.

10. The system of claim 9, wherein the insert further includes a rigid plate.

11. The system of claim 10, wherein the rigid plate further includes a second portion of the third hook and loop fastener.

12. The system of claim 8, wherein when the insert is inserted in the opening, the first portion and the second portion of the third hook and loop fastener connect.

13. A first aid kit for rapid access, comprising:
a pouch including a rear panel, a first opposing side, a second opposing side, each of the opposing sides including a first edge and a second edge, each of the first and second opposing sides being attached to the rear panel along the first edge, a front panel including a first portion of a first hook and loop fastener, the front panel being attached, at least in part, to each of the first and second opposing sides along the second edge, the rear panel, the first and second opposing sides, and the front panel forming an interior;
an interior flap attached to the rear panel, the interior flap including a second portion of the first hook and loop fastener, the second portion of the first hook and loop fastener removably attached to the first portion of the first hook and loop fastener;
an interior end tab attached to the rear panel and the interior flap, the interior end tab including a first portion of a second hook and loop fastener;
an exterior end tab attached along a single edge of the exterior end tab to the front panel, the exterior end tab including a second portion of the second hook and loop fastener, the second portion of the second hook and loop fastener removably attached to the first portion of the second hook and loop fastener;
a first zipper including a first tape portion and a second tape portion, the first tape portion connected to the front panel, and the second tape portion connected along the second edge of the first opposing side;
a second zipper including a first tape strip and a second tape strip, the first tape strip connected to the front panel and the second tape strip connected along the second edge of the second opposing side;
an insert removably placed in the interior, the insert including a removal strap for withdrawing the insert from the interior.

14. The system of claim 13, wherein the rear panel further includes a rigid plate.

15. The system of claim 14, wherein the rigid plate further includes a first portion of a third hook and look fastener.

16. The system of claim 15, wherein the rear panel includes a section portion of the third hook and loop fastener.

17. The system of claim 13, further including a ripcord, which when pulled, separates the first hook and look fastener and the second hook and loop fastener, and unzips the first zipper and the second zipper.

18. The system of claim 17, wherein the insert further includes a removal strap accessible after the ripcord is pulled.

19. The system of claim 13, wherein the exterior end tab is attached to the front panel only along a single edge.

20. The system of claim 13, wherein the interior flap is attached to the rear panel only along a single edge.
